(19) United States
(12) Patent Application Publication
(10) Pub. No.: US 2022/0233167 A1
(43) Pub. Date: Jul. 28, 2022

(54) DETECTING PATHOLOGIES USING AN ULTRASOUND PROBE

(71) Applicant: Echo Mind AI Corp, Highlands Ranch, CO (US)

(72) Inventors: LEO MAX HARKER, HIGHLANDS RANCH, CO (US); DARREN S. LUND, PROVO, UT (US); CASEY KIANE CHARLEBOIS, CLINTON TOWNSHIP, MI (US); Manuel Rodrigo Parra Castaneda, Chicago (CL)

(21) Appl. No.: 17/580,916

(22) Filed: Jan. 21, 2022

Related U.S. Application Data
(60) Provisional application No. 63/140,779, filed on Jan. 22, 2021.
(60) Provisional application No. 63/273,186, filed on Oct. 29, 2021.

(51) Int. Cl. A61B 8/00; A61B 8/08

(52) U.S. Cl. CPC A61B 8/4444; A61B 8/5215; A61B 8/461

(57) ABSTRACT

The present application describes a musculoskeletal diagnosis system that receives, in real-time or substantially real-time, various objects from a musculoskeletal ultrasound probe. Once the objects are received, the musculoskeletal diagnosis system analyzes the various objects to detect various pathologies and direct additional movement of the ultrasound probe as subsequent objects are taken.

### CROSS-REFERENCE TO RELATED APPLICATIONS

The present application is a non-provisional application of, and claims priority to, U.S. Provisional Patent Application No. 63/140,779, filed Jan. 22, 2021 and entitled “Detecting Pathologies Using an Ultrasound Probe” and claims priority to U.S. Provisional Patent Application No. 63/273,186, filed Oct. 29, 2021 and entitled “Detecting Pathologies Using an Ultrasound Probe”, the entire disclosures of which are hereby incorporated by reference in their entirety.

### BACKGROUND

An ultrasound probe may be used to enable a doctor to detect a number of different musculoskeletal conditions such as tears, strains, and the like. However, doctors are typically not trained to use an ultrasound probe and, as such, refer their patients to an ultrasound technician. Once a patient is referred to the ultrasound technician, the patient must schedule an appointment with the ultrasound technician and subsequently schedule a follow-up appointment with the doctor. This process can take a number of weeks.

### SUMMARY

The present application describes a musculoskeletal diagnosis system that receives, in real-time or substantially real-time, various objects from a musculoskeletal ultrasound probe and analyzes the various objects to detect various pathologies. The musculoskeletal diagnosis system may also direct movement of the ultrasound probe as subsequent objects are taken. The musculoskeletal diagnosis system enables a healthcare provider, such as a doctor, nurse etc., to capture ultrasound objects “in-house” (e.g., without the need of referring her patients to a specialized ultrasound technician or a sonographer). Additionally, by following real-time instructions provided by the musculoskeletal diagnosis system, the doctor may capture objects that may be more conducive to providing an accurate diagnosis.

Accordingly, aspects of the present disclosure describe a method that includes receiving an object from an ultrasound probe. Once the object is received, the object is compared to a series of stored objects. Based on comparing the object to the series of stored objects, a location of the ultrasound probe with respect to a patient is determined. An orientation of the ultrasound probe with respect to the patient may also be determined. Based on the location of the ultrasound probe and/or the orientation of the ultrasound probe, a movement instruction for the ultrasound probe is generated. In an example, the movement instruction, when followed, may be used to capture an additional object of an area of interest. The additional object may be used to better diagnose a potential problem in the area of interest when compared to the originally captured/received object. The movement instruction is then provided, in real-time or substantially real-time, on a display of a computing device.

The present disclosure also describes a system comprising a processor and a memory communicatively coupled to the processor. The memory stores instructions that, when executed by the processor, perform operations. These operations may include receiving an object of an area of interest from an ultrasound probe. Once the object is received, an object analysis process is executed on the object. Based on a result of the object analysis process, a movement instruction for the ultrasound probe is generated. The movement instruction provides real-time instructions regarding how and/or where to move the ultrasound probe to capture an additional object of the area of interest. The movement instruction may then be output on a display of a computing device.

Also described is a method that includes receiving an object from an ultrasound probe and analyzing the object to identify a pathology in the object. A movement instruction for the ultrasound probe is generated. In an example, the movement instruction is based, at least in part, on the pathology. The movement instruction may then be provided on a display of a computing device in real-time or substantially real-time. Movement of the ultrasound probe may subsequently be detected. Based on detecting the movement of the ultrasound probe, an updated movement instruction is provided in real-time or substantially real-time. The updated movement instruction is provided on the display of the computing device in real-time or substantially real-time.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Non-limiting and non-exhaustive examples are described with reference to the following Figures.

FIG. 1A illustrates an example musculoskeletal diagnosis system according to an example.

FIG. 1B illustrates the musculoskeletal diagnosis system of FIG. 1A in which movement instructions may be provided to an ultrasound probe and/or to a computing device associated with the ultrasound probe and in which a diagnosis of a detected pathology is provided to the computing device according to an example.

FIG. 2 illustrates a method for providing movement instructions for an ultrasound probe according to an example.

FIG. 3 illustrates a method for providing a diagnosis of a detected pathology according to an example.

FIG. 4 is a block diagram illustrating example physical components of a computing device with which aspects of the disclosure may be practiced.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Examples may be practiced as methods, systems or devices. Accordingly, examples may take the form of a hardware implementation, an entirely software implementation, or an implementation combining software and hardware aspects. The following detailed description is therefore not to be taken in a limiting sense, and the scope of the present disclosure is defined by the appended claims and their equivalents.

A musculoskeletal ultrasound probe may be used to enable a doctor to detect a number of different musculoskeletal conditions such as tears, strains, fractures and the like. Additionally, an ultrasound probe may allow the doctor to detect various pathologies such as arthritis, bursitis, osteoarthritis, tendinitis, etc. However, as indicated above, doctors are typically not trained to use an ultrasound probe. As such, the doctor typically refers her patients to an ultrasound technician or a sonographer. The ultrasound technician takes a number of different images of an area of interest (e.g., a knee of the patient) and returns the images back to the doctor since the ultrasound technician is typically not trained/qualified to provide a diagnosis. The patient may then be required to schedule a follow-up visit with the doctor to receive the diagnosis. This process can take a number of weeks.

Additionally, once the doctor has received the images, the doctor may need to visually identify a particular musculoskeletal condition. The accuracy of the doctor's diagnosis may be heavily reliant on the doctor's own experience and what the doctor can visually see when looking at the images. The quality of the received images and/or an angle from which the images were captured may impact the doctor's diagnosis.

In order to address the above, the present application describes a musculoskeletal diagnosis system that receives ultrasound and/or doppler images and/or other such data (collectively referred to herein as “objects”) from an ultrasound probe and analyzes the objects to identify different pathologies (e.g., fractures, effusions, bursitis, dislocations, arthritis, tendon injuries, blood flow, etc.) in various musculoskeletal areas (e.g., tendons, muscles, ligaments, bones, etc.) of a patient from which the ultrasound probe captured the objects.

The above may be accomplished by utilizing a training system that effectively learns to recognize what is shown in various objects. For example, during a training phase of the musculoskeletal diagnosis system, the training system of the musculoskeletal diagnosis system may receive various objects of different musculoskeletal and/or anatomical structures as well as various pathologies that may be present or otherwise detected in these structures. As the objects are received, the training system analyzes each object and identifies certain characteristics within the object. The characteristics may be used to ultimately identify the musculoskeletal and/or anatomical structure as well as any identifiable pathologies. In some examples, feedback corresponding to an analyzed object may be received (e.g., from a doctor, a physician, a sonographer, another artificial intelligence program or entity, etc.) that indicates whether the analysis was correct. In some examples, the feedback may be received in real-time or substantially real-time. Thus, over time, the musculoskeletal diagnosis system is able to refine the identification and diagnosis process.

The musculoskeletal diagnosis system also generates and/or provides movement instructions to an operator of the ultrasound probe in real-time or substantially real-time. Thus, the operator of the ultrasound probe may learn how to use the ultrasound probe and also how to capture subsequent objects that may assist the musculoskeletal diagnosis system in accurately diagnosing the pathology. Once the condition is diagnosed, the musculoskeletal diagnosis system may provide the diagnosis to the operator of the ultrasound probe. In an example, the movement instructions and/or the diagnosis may be provided on a display of a computing device.

In yet another example, the musculoskeletal diagnosis system may guide and/or provide instructions to a user regarding a desired starting point for the ultrasound probe. Once the starting point has been located and the musculoskeletal diagnosis system confirms or otherwise verifies that the ultrasound probe has been placed at the location, the musculoskeletal diagnosis system may provide movement instructions for the ultrasound probe.

For example, the location of the ultrasound probe may be verified based on one or more landmarks. The one or more landmarks may be identified by analyzing objects captured by the ultrasound probe and comparing those objects to one or more known musculoskeletal features of the body on which the ultrasound probe is placed. For example, a shoulder of an individual may have a particular musculoskeletal landmark and a knee of the individual may have a different musculoskeletal landmark. Once a landmark is identified, the location of the ultrasound probe may be determined/verified.

Movement instructions may then be provided. In one example, the movement instructions may be predetermined or pre-prescribed based, at least in part, on a starting point or other location of the ultrasound probe on the body of the individual.

As the ultrasound probe moves in the pre-prescribed motions, a series of objects may be captured in real-time or substantially real-time. The series of objects may be analyzed and/or color coded such as described below.

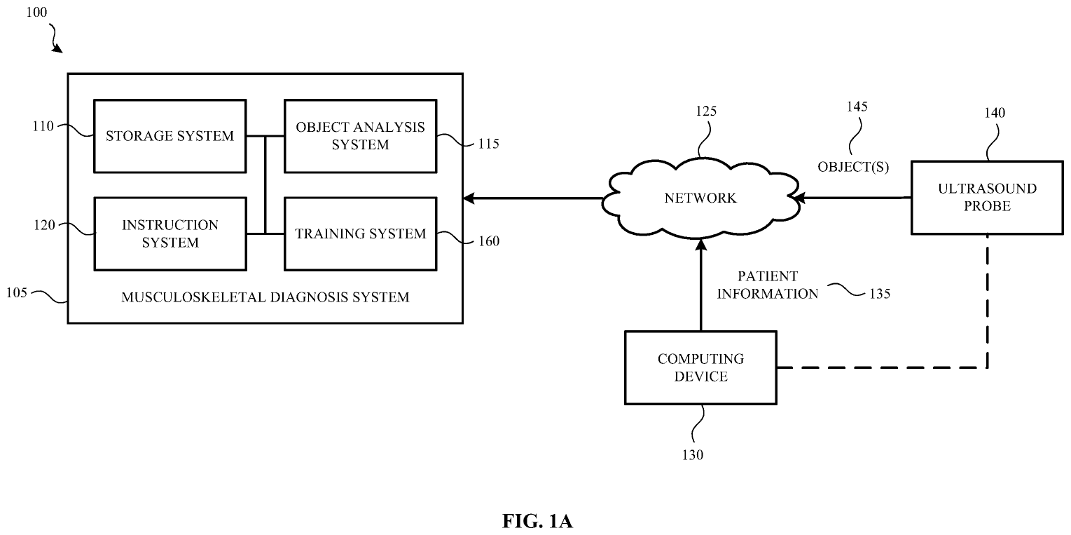

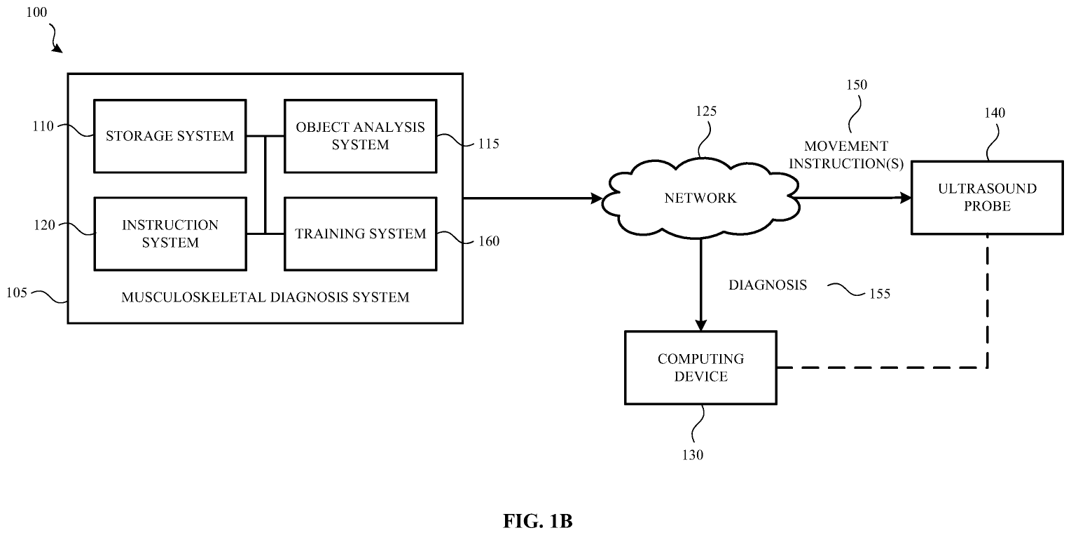

These and other examples will be shown and described in more detail with respect to FIGS. 1A-FIG. 3 below.

FIG. 1A illustrates a system 100 for providing movement instructions and/or a pathology diagnosis based on one or more objects received from an ultrasound probe according to an example. Although the examples described herein are directed to musculoskeletal diagnoses, the concepts described herein may have any number of different applications in which ultrasound objects are used to diagnose various conditions.

The system 100 includes a musculoskeletal diagnosis system 105. The musculoskeletal diagnosis system 105 may be communicatively coupled to an ultrasound probe 140 via a network 125. The musculoskeletal diagnosis system 105 may also be communicatively coupled to a computing device 130 via the network 125. Although a network 125 is specifically shown and described, the musculoskeletal diagnosis system 105 may be communicatively coupled to the ultrasound probe 140 and/or to the computing device 130 through various communication protocols including, but not limited to, Bluetooth, near-field communication, or other wireless (or wired) communication protocols.

Additionally, although the musculoskeletal diagnosis system 105 is shown as being a separate system from the computing device 130, the musculoskeletal diagnosis system 105 may be part of or otherwise integrated with the computing device 130. Likewise, the musculoskeletal diagnosis system 105 may be integrated or otherwise associated with the ultrasound probe 140. Although one computing device 130 and one ultrasound probe 140 are shown and described, the system 100 may include any number of ultrasound probes 140 and/or computing devices 130.

The musculoskeletal diagnosis system 105 may include a storage system 110, an object analysis system 115, a training system 160 and an instruction system 120. The storage system 110 may store various training and/or reference objects. The training and/or reference objects may be objects in which various pathologies and/or other issues (e.g., tears, fractures) have been diagnosed or otherwise identified. The training and/or reference objects may also include information regarding the captured musculoskeletal area (e.g., knee, elbow, ankle).

In another example, the storage system 110 may include landmark objects that are used by the musculoskeletal diagnosis system 105 determine a location of the ultrasound probe 140 on a body of an individual. For example, a musculoskeletal system of an individual includes various bony landmarks. As the ultrasound probe 140 captures objects, the captured objects may be compared with the stored landmark objects to help determine whether the ultrasound probe is placed at a desired location (e.g., knee, elbow, shoulder, or ankle) in which the bony landmarks match the landmark objects. If the ultrasound probe 140 is placed at the desired location, the instruction system 120 may provide movement instructions to the computing device 130 and/or otherwise begin capturing objects 145 such as will be described in greater detail below.

The landmark objects, the training objects and/or the reference objects may be provided to the training system 160. As objects are received, the training system 160 “learns” or otherwise generates an analysis process that identifies one or more characteristics about the object. Using the characteristics, the training system 160 learns various structures, features, views, outlines etc. of different musculoskeletal and/or anatomical structures. In some examples, the objects that are received by the training system 160 include information about the musculoskeletal and/or anatomical structures. This information may include identifying information about a specific musculoskeletal and/or anatomical structure in an object (e.g., landmarks), an identified pathology, an identified view or angle from which the object was taken etc. In another example, the information described above may be determined by the training system 160. As the training system 160 analyzes the objects and provides its analysis, feedback may be provided to the training system regarding whether the analysis is correct. For example, a doctor, technician, sonographer etc. may provide feedback about a particular object that was used in by the training system indicating whether the analysis of a pathology and/or whether an identified musculoskeletal and/or anatomical structure is correct.

In another example, the training system 160 may receive feedback regarding a matching process between the stored landmark objects and the captured bony landmark objects to help ensure the musculoskeletal diagnosis system 105 is properly identifying a current location of the ultrasound probe 140 on the body of the individual.

Although the training system 160 may be trained using various training objects, the training system 160 may also receive subsequent objects (e.g., those that are captured by an ultrasound probe 140), provide a diagnosis such as will be described in more detail below, and receive feedback about its diagnosis (e.g., either a correct diagnosis/identification or an incorrect diagnosis/identification) from a doctor or other healthcare provider. Although feedback from a doctor is specifically mentioned, it is contemplated that the feedback may be received from another artificial intelligence system that has been trained to recognize various musculoskeletal and/or anatomical structure and/or various pathologies. Thus, the musculoskeletal diagnosis system 105 may continually improve its ability to provide accurate diagnoses and/or location information.

The various objects may be received from a number of different individuals. In an example, the individual from which the object was captured has provided consent that the objects can be used. Any personal information associated with individual may be removed and the objects may be securely stored.

As discussed above, the storage system may store various objects of musculoskeletal areas. For example, the storage system 110 may store various different objects of a knee. Additionally, each object may be associated with a particular pathology. For example, a first series of objects may show a number of different meniscus tears in various knees while a second series of objects may show the effects of bursitis in various knees.

Although objects are specifically mentioned and described, various types of objects and/or data received from the ultrasound probe 140, either alone or in combination, may be used to determine a pathology, identify and/or color code various anatomical structures, provide movement instructions, or otherwise help and/or assist in determining a diagnosis. In an example, the data may include ultrasound waves. As the musculoskeletal diagnosis system 105 receives the ultrasound waves, a speed at which the waves travel or is otherwise moving may assist the musculoskeletal diagnosis system 105 in determining a position of the ultrasound probe 140, a direction in which the ultrasound probe 140 may need to move, a position of the ultrasound probe 140 and so on.

In other examples, the ultrasound waves may assist the musculoskeletal diagnosis system 105 in determining a pathology or otherwise providing a diagnosis. In an example, the ultrasound waves may be raw ultrasound data such as, but not limited to, RF data, IQ data, B mode data and the like. The data may also include three-dimensional data that assists in reconstructing one or more objects in a three-dimensional format and/or assist the operator by providing movement instructions.

When the musculoskeletal diagnosis system 105 receives an object 145 from the ultrasound probe 140, the object analysis system 115 may analyze the object 145 using the analysis process described above. In an example, the analysis process may compare the received object 145 with one or more stored objects in order to identify a pathology in the object 145. For example, if the object 145 is an object of a knee, the object analysis system may determine that the patient has a meniscus tear based on comparing the object 145 to the stored objects of knees with meniscus tears.

In another example, the object analysis system 115 may also compare the object 145 to various stored objects (e.g., landmark objects) to identify the musculoskeletal area captured in the object 145 (e.g., whether the object is a knee, a tendon in the knee, a shoulder, etc.). In yet another example, the object analysis process may analyze the object to identify various characteristics within the object. Based on the characteristics within the object an using information learned in the training process by the training system 160, provide an identification of the musculoskeletal area and/or a pathology.

In another example, an individual operating the ultrasound probe 140 and/or the computing device 130 may provide an input that identifies the musculoskeletal area contained in the captured object 145. The individual operating the ultrasound probe 140 may also provide patient information 135 to the musculoskeletal diagnosis system 105. The patient information 135 may include demographic information associated with the patient. The demographic information may be used by the object analysis system 115 to further refine a diagnosis of the pathology and/or the musculoskeletal area. For example, demographic information may be used to identify stored objects from individuals whose demographics match or otherwise correspond to one or more demographics of the individual associated with the object 145.

When the object 145 is received by the musculoskeletal diagnosis system 105, the object analysis system 115 may compare one or more patterns and/or shapes in the object 145 to one or more patterns and/or shapes in the stored objects to identify the pathology and/or a musculoskeletal area on which the ultrasound probe 140 is placed. In another example, the object analysis system 115 may compare a determined or identified echogenicity within the object to an echogenicity of comparable musculoskeletal areas. In yet another example, the object analysis system 115 may compare a determined or identified echotexture of the captured musculoskeletal area within the object 145 to an echotexture of comparable musculoskeletal areas in the stored objects in order to identify a pathology or other potential issue.

In another example, when various characteristics, patterns, shapes, echogenicity, echotexture etc. are identified in the object 145, the object analysis system 115 may be able to identify the pathology and/or musculoskeletal area based on the previously described “learned” information. Although specific examples have been given, the object analysis system 115 may use any number of machine learning and/or artificial intelligence techniques in order to provide a preliminary diagnosis of the pathology in the object 145 including but not limited to, object recognition, pattern matching, prediction algorithms and the like.

The object analysis system 115 may also determine movement and/or position instructions for the ultrasound probe 140. The movement and/or position instructions may be based on comparing the object 145 to one or more previously captured/stored objects. For example, if the object 145 is an object of a top of the knee of the patient and the object analysis system 115 detects a particular pathology, the object analysis system 115 may determine, based, at least in part, on comparing the object 145 with various stored objects, that if the ultrasound probe 140 is moved in a certain direction and/or to a particular position on the patient's knee (and/or moved in a particular orientation with respect to the patient's knee) an additional object of the area of interest could be captured. The additional object may be an object that increases a likelihood that the object diagnosis system 115 will correctly or otherwise accurately identify the pathology (e.g., more accurately diagnose the pathology/condition when compared with the diagnosis that is based on the captured object 145).

In another example, the movement and/or position instructions may be determined based on information learned by the musculoskeletal diagnosis system 105 and/or the training system 160. For example, during a training phase, the training system 160 may learn how various views a musculoskeletal area can be obtained in order to better identify the area and/or diagnosis a detected pathology. In some examples, feedback about movement and/or position instructions may be provided in real-time or substantially real-time (e.g., by a doctor, sonographer, etc.). For example, and referring to FIG. 1B, FIG. 1B illustrates the musculoskeletal diagnosis system 105 of FIG. 1A in which movement instructions 150 are provided to the ultrasound probe 140 and/or to the computing device 130 associated with the ultrasound probe 140. Additionally, FIG. 1B shows that a diagnosis 155 of a detected pathology may be provided to the computing device 130.

In order to generate and/or provide movement instructions 150, the musculoskeletal diagnosis system 105 may receive position information and/or orientation information from the ultrasound probe 140. For example, the ultrasound probe 140 may include an accelerometer, a gyroscope or other such positioning system that indicates an angle, position and/or orientation of the ultrasound probe 140 with respect to the patient on which ultrasound probe 140 is placed.

This information may be provided to the musculoskeletal diagnosis system 105 as part of the object 145. Thus, when the object analysis system 115 receives the position/orientation information, the object analysis system 115, in combination with the instruction system 120, may determine and/or generate movement instructions 150. The movement instructions 150, when followed by an operator of the ultrasound probe, cause the ultrasound probe 140 to move in a direction/orientation and/or to a location to capture additional objects. The object analysis system 115 and/or the instruction system 120 may determine, based on the previously described comparison and/or analysis process, that the additional objects, when captured while the ultrasound probe is moving to and/or is at the designation location, will assist the object analysis system 115 in accurately identifying the pathology.

In another example, the instruction system 120 may include predetermined and/or preset movement instructions that are provided to the ultrasound probe 140 based on, for example, a determined or otherwise identified location of the ultrasound probe 140. These movement instructions may be specific to a particular portion or area of the body on which the ultrasound probe 140 is placed. For example, the storage system 110 may include a first movement instruction or set of movement instructions when the ultrasound probe 140 is placed at a first starting point (e.g., a knee of an individual) and a second movement instruction or set of movement instructions when the ultrasound probe 140 is placed on a second starting point (e.g., a shoulder of the individual).

Although the movement instructions 150 are shown in FIG. 1B as being provided to the ultrasound probe 140, the movement instructions 150 may also be provided to the computing device 130. The movement instructions may be provided to the ultrasound probe 140 and/or to the computing device 130 is real-time or substantially real-time. The movement instructions 150 may include various visual indicators (e.g., arrows, animations, colors, text, or any combination thereof) on a display of the computing device 130 and/or on a display associated with the ultrasound probe 140. As explained above, the movement instructions assist the operator in moving the ultrasound probe 140 to the desired location and/or orientation. In an example, the movement instructions 150 may also cause a haptic output and/or an audible notification/alarm to be output by the ultrasound probe 140 and/or the computing device 130 in order to provide additional real-time feedback to the operator.

The object analysis system 115 may also provide a diagnosis 155 to the computing device 130. The diagnosis 155 may include an object of the musculoskeletal area and may include a detected pathology or other potential issue identified by the object analysis system 115. In an example, the diagnosis 155 may be associated with a confidence threshold as to the accuracy of the diagnosis 155. In some examples, the diagnosis 155 may be provided to the computing device 130 in real-time or substantially real-time.

The confidence threshold may be updated and/or displayed in real-time or substantially real-time. Thus, as additional objects are captured (e.g., based on the operator following the provided movement instructions 150) the musculoskeletal diagnosis system 105 may update the confidence threshold as additional objects are received. Once a predetermined maximum confidence threshold is reached (e.g., the musculoskeletal diagnosis system 105 can determine with a 90% confidence score that the diagnosis 155 is accurate) the musculoskeletal diagnosis system 105 may stop providing movement instructions 150.

In an example, the diagnosis 155 may include one or more of the captured objects. The one or more captured objects may be selected, by the object analysis system 115, as having the highest quality when compared to the other captured objects (e.g., the objects included in the diagnosis are those objects in which the pathology is clearly defined and/or shown). In another example, the diagnosis 155 may include one or more objects (e.g., a series of objects) that include optimal objects (e.g., the objects that provide the most detail when compared with the other captured objects in the series of objects). The diagnosis 155 may include or otherwise be associated with a color scheme. Each color in the color scheme may be associated with a particular pathology.

For example, the diagnosis 155 may show a meniscus tear may by a blue highlight, a blue circle or other visual indicator. In another example, the diagnosis 155 may show bursitis using a yellow highlight, circle or other visual indicator.

In an example, a shading/opacity of the visual indicator associated with the particular pathology may be based on a confidence level associated with the diagnosis. For example, an identified area of interest in the diagnosis 155 may have a first opacity if the musculoskeletal diagnosis system 105 has a first confidence level that the diagnosis is correct. In another example, an identified area of interest in the diagnosis 155 may have a second opacity if the musculoskeletal diagnosis system 105 has a second confidence level (e.g., a higher confidence level when compared to the first confidence level) that the diagnosis is correct.

In yet another example, a shading, highlight and/or color of the visual indicator associated with the particular pathology or other detected issue may be based, at least in part, on a determined severity. For example, red visual indicator that identifies a small meniscus tear may be semi-transparent while a red visual indicator that identifies a larger meniscus tear may be more opaque.

As each diagnosis 155 is determined, various objects associated with the diagnosis 155 may be securely stored by the storage system 110. As such, the musculoskeletal diagnosis system 105 may use the objects to refine its diagnosis and instruction generation process.

In yet another example, the object analysis system 115 may also be able to recognize or otherwise identify various anatomical structures in various objects 145 (FIG. 1A). For example, as an object 145 is received by the musculoskeletal diagnosis system 105, the object analysis system 115 may perform object processing, object comparison (e.g., comparing the object 145 with one or more stored objects) and/or machine learning on the object to identify an anatomical structure in the object 145. In an example, this analysis may include determining or otherwise identifying various variables from ultrasound data associated with the object 145 to code or otherwise identify the anatomical structure.

Once an anatomical structure is identified, the object analysis system 115 may color code various anatomical structures and provided the color coded anatomical structures as part of the diagnosis. In an example, an anatomical structure may be color coded in a first color and a detected issue with the anatomical structure (e.g., a tear, sprain etc.) may be color coded or otherwise identified in a second color such as described above.

The musculoskeletal system 105 may also combine various object processing techniques in order to identify various anatomical/musculoskeletal structures and/or to identify various pathologies. The anatomical/musculoskeletal structures and/or the pathologies may be color coded such as described above.

For example and referring back to FIG. 1A, the object analysis system 115 may receive an object 145 (e.g., from an ultrasound probe 140 or other source), analyze the object 145 to determine an outline of a structure (e.g., a bone) using a first object processing technique and/or data and use a second object processing technique (e.g., various viewpoints or other information from an ultrasound) to create a more complete view of the anatomical/musculoskeletal structures and/or the pathologies than would be possible if the objects were analyzed separately. The various parts of the finalized object may be color coded based on the determine anatomical structure and/or pathology such as described above.

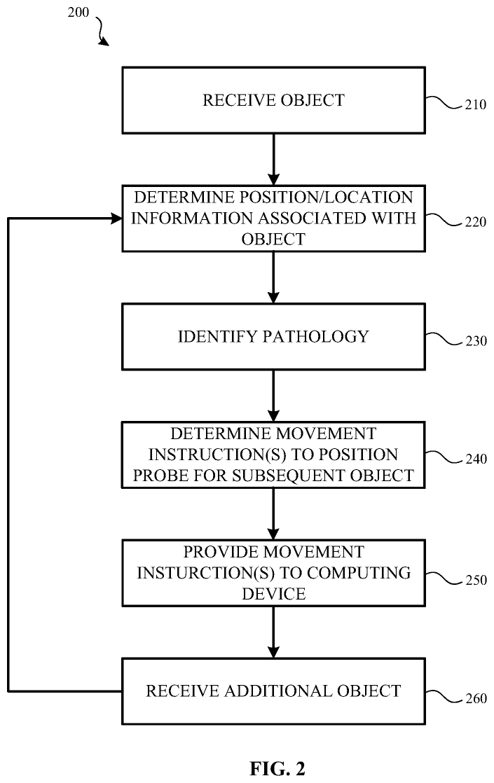

FIG. 2 illustrates a method 200 for providing movement instructions for an ultrasound probe according to an example. The method 200 may be used or otherwise performed by the musculoskeletal diagnosis system 105 shown and described with respect to FIG. 1A and FIG. 1B.

Method 200 begins when the musculoskeletal diagnosis system receives (210) an object. The object may be received from an ultrasound probe such as described above. In another example, the object may be received from a computing device associated with an ultrasound probe. In yet another example, the musculoskeletal diagnosis system resides in the cloud or is otherwise remote from an apparatus that captures ultrasound objects. As such, various doctors, nurses, ultrasound technicians or other healthcare providers may send various captured objects to the musculoskeletal diagnosis system via a network or other communication protocol.

In another example, an operator of the ultrasound probe may manually (or otherwise) provide a current location of the ultrasound probe on the body of the individual. In this example, the captured object may be used to verify that the ultrasound probe is placed at the proper/designated location.

Once the object is received, an object analysis system of the musculoskeletal diagnosis system determines (220) or otherwise identifies position/location/orientation information associated with the object. In an example, the position/location/orientation information may be determined by comparing the captured object to one or more stored objects. In another example, the position/location/orientation information may be provided by the ultrasound probe. In yet another example, the position/location/orientation information may be provided by an operator of the ultrasound probe.

The object analysis system may also identify (230) a pathology or other potential issue in the captured object. The pathology may be detected or otherwise identified by comparing various textures, patterns, etc. within the captured object to various textures, patterns, etc. of the stored objects such as described above. In another example, the pathology is identified by an analysis process generated or otherwise learned by a training system such as described above.

Once the pathology is identified, an instruction system of the musculoskeletal diagnosis system may determine (240) movement instructions for the ultrasound probe. The movement instructions, when followed by the operator of the ultrasound probe, may cause the ultrasound probe to be positioned or otherwise moved in a direction that enables the musculoskeletal diagnosis system to a more accurately diagnose the pathology (e.g., enable the musculoskeletal diagnosis system to more accurately diagnose the pathology using the additional captured objects when compared with a diagnosis that is based on the initial captured object).

In an example, the movement instructions may be specific to the area of the body on which the ultrasound probe is placed. For example, if the ultrasound probe is placed on the knee of the individual, a first set of movement instructions may be provided. Likewise, if the ultrasound probe is placed on a shoulder of the individual, a second set of movement instructions may be provided. In some examples, the first and/or second set of movement instructions may be pre-determined.

When the movement instructions are generated or otherwise identified, the movement instructions are provided (250) to the ultrasound probe and/or to an associated computing device. In an example, the movement instructions may be provided to the computing device in real-time or substantially real-time. The movement instructions may include various visual and/or audio indicators (e.g., color-coded arrows, animations, haptic feedback, alarms, sounds, etc.) to enable the operator to easily follow the movement instructions. The movement instructions may also help the operator learn how to use the ultrasound probe since the operator is receiving real-time instructions. As the operator follows the movement instructions, additional objects may be received (260) and the method 200 may be repeated.

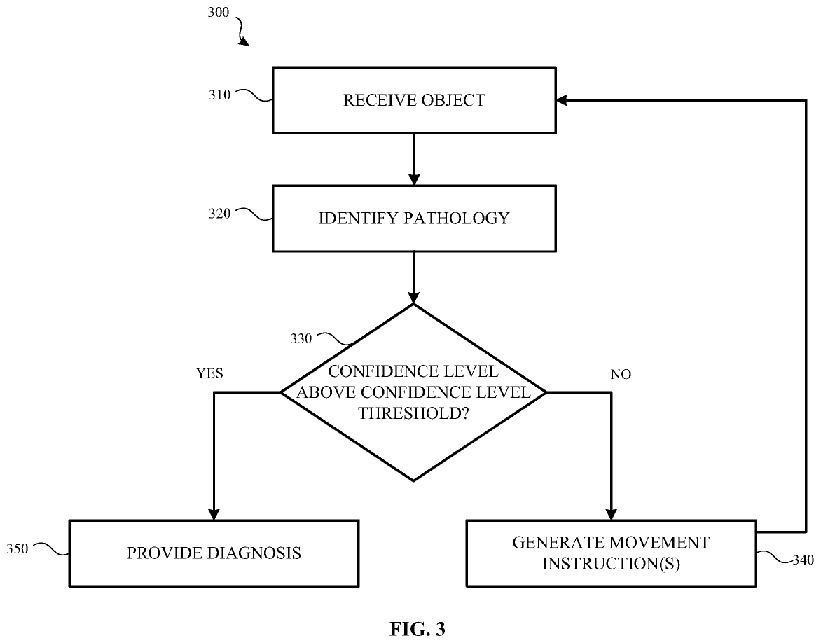

FIG. 3 illustrates a method 300 for providing a diagnosis of a detected pathology according to an example. In an example, the method 300 may be used or otherwise performed by the musculoskeletal diagnosis system 105 shown and described with respect to FIG. 1A and FIG. 1B. Additionally, the method 300, or portions of the method 300, may be combined with various operations shown and described with the method 200 of FIG. 2.

Method 300 begins when the musculoskeletal diagnosis system receives (310) an object. The object may be received from an ultrasound probe or from a computing device such as described above. Once the object is received, the object is provided to an object analysis system. The object analysis system analyzes the object using a learned object analysis process such as described above in order to identify (320) a pathology. In another example, the object analysis system may compare the object with one or more captured objects to identify a pathology.

The musculoskeletal diagnosis system may then determine (330) a confidence score associated with the identified pathology. For example, by comparing textures, patterns, etc. in the received object with various textures, patterns, etc. of the captured objects, the musculoskeletal diagnosis system may determine a confidence level for its diagnosis.

For example, the musculoskeletal diagnosis system may determine, based on the comparison of objects, using the analysis process and/or from feedback provided by a healthcare provider, that it is sixty percent confident its diagnosis is accurate. The confidence level is then compared to confidence level threshold. The confidence level threshold may indicate how confident the musculoskeletal diagnosis system needs to be in order to finish or otherwise provide a final diagnosis. In an example, the confidence threshold may by seventy percent or more, eighty percent or more, or ninety percent or more. Although specific thresholds are mentioned, any threshold may be used.

If it is determined the confidence level of the diagnosis is not above the confidence level threshold, movement instructions are generated (340) and provided to the ultrasound probe and/or computing device such as previously described. As the ultrasound probe is moved, additional objects are subsequently captured and/or received (310). However, if it is determined that the confidence level is above the confidence level threshold, the musculoskeletal diagnosis system may provide (350) a diagnosis such as described above.

In an example, an entire series of captured objects may be used to determine the diagnosis. However, the musculoskeletal diagnosis system may determine that a particular object or series of objects may be used to best illustrate the determine diagnosis. As such, the musculoskeletal diagnosis system may identify and/or color code particular frames (sequential and/or non-sequential frames) and provide the particular frames to a computing device of the individual and/or the doctor. As the frames are being displayed, the musculoskeletal diagnosis system may automatically skip to the frames that have been color coded and/or otherwise been identified as being optimal frames when compared with other captured objects.

In another example, the musculoskeletal diagnosis system may analyze one or more objects in the series of captured objects and determine which objects have landmarks or other features that may be readily identifiable by the individual and/or the doctor. In such an example, the musculoskeletal diagnosis system may enable the individual and/or the doctor to provide input that causes these objects to be provided on a display. In another example, the musculoskeletal diagnosis system may automatically skip ahead to the objects that have the readily identifiable landmarks or other features. In some examples, the doctor and/or individual may provide parameters or other input that causes objects or frames with those parameters the be identified and subsequently presented on a display.

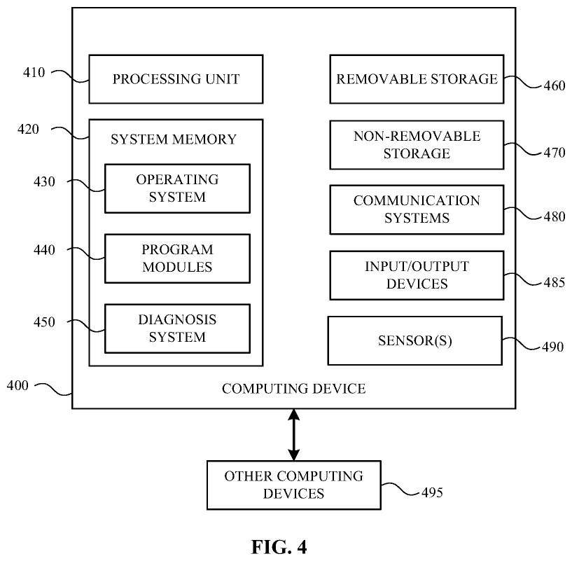

FIG. 4 and its associated descriptions provide a discussion of an example computing device that may be used with the various systems described herein. However, the illustrated computing device is an example and is not limiting as a vast number of electronic device configurations may be utilized for practicing various aspects of the disclosure.

FIG. 4 is a block diagram illustrating physical components (e.g., hardware) of a computing device 400 with which aspects of the disclosure may be practiced. The computing device 400 may be integrated or otherwise associated with any of the various systems described above with respect to FIG. 1A and FIG. 1B. For example, the computing device 400 may be integrated or otherwise associated with an ultrasound probe, the musculoskeletal diagnosis system 105, the computing device 130, the object analysis system 115, the instruction system 120 and/or the storage system 110.

In a basic configuration, the computing device 400 may include at least one processing unit 410 and a system memory 420. Depending on the configuration and type of computing device, the system memory 420 may comprise, but is not limited to, volatile storage (e.g., random access memory), non-volatile storage (e.g., read-only memory), flash memory, or any combination of such memories. The system memory 420 may include an operating system 430 and one or more program modules 440 or components suitable for performing the various operations described above. The operating system 430 may be suitable for controlling the operation of the computing device 400. The system memory 420 may include a diagnosis system 450.

The computing device 400 may have additional features or functionality. For example, the computing device 400 may also include additional data storage devices (removable and/or non-removable) such as, for example, magnetic disks, optical disks, or tape. Such additional storage is illustrated in FIG. 4 by a removable storage device 460 and a non-removable storage device 470.

As stated above, a number of program modules 440 and data files may be stored in the system memory 420. While executing on the processing unit 410, the program modules 440 may perform the various processes including, but not limited to, the aspects, as described herein.

Furthermore, examples of the disclosure may be practiced in an electrical circuit comprising discrete electronic elements, packaged or integrated electronic chips containing logic gates, a circuit utilizing a microprocessor, or on a single chip containing electronic elements or microprocessors. For example, examples of the disclosure may be practiced via a system-on-a-chip (SOC) where each or many of the components illustrated in FIG. 4 may be integrated onto a single integrated circuit. Such an SOC device may include one or more processing units, graphics units, communications units, system virtualization units and various application functionality all of which are integrated (or “burned”) onto the chip substrate as a single integrated circuit.

When operating via an SOC, the functionality, described herein, with respect to the capability of client to switch protocols may be operated via application-specific logic integrated with other components of the computing device 400 on the single integrated circuit (chip). Examples of the disclosure may also be practiced using other technologies capable of performing logical operations such as, for example, AND, OR, and NOT, including but not limited to mechanical, optical, fluidic, and quantum technologies. In addition, examples of the disclosure may be practiced within a general-purpose computer or in any other circuits or systems.

The computing device 400 may also have one or more input/output device(s) 485. These include, but are not limited to, a keyboard, a trackpad, a mouse, a pen, a sound or voice input device, a touch, force and/or swipe input device, a display, speakers, a printer, etc. The aforementioned devices are examples and others may be used. The computing device 400 may include one or more communication systems 480 that allow or otherwise enable the computing device 400 to communicate with remote computing devices 495. Examples of suitable communication connections include, but are not limited to, radio frequency (RF) transmitter, receiver, and/or transceiver circuitry; universal serial bus (USB), parallel, and/or serial ports.

The computing device may include one or more sensors 490. The sensors may include location sensors, accelerometers, position sensors the like.

The term computer-readable media as used herein may include computer storage media. Computer storage media may include volatile and nonvolatile, removable and non-removable media implemented in any method or technology for storage of information, such as computer readable instructions, data structures, or program modules.

The system memory 420, the removable storage device 460, and the non-removable storage device 470 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 400. Any such computer storage media may be part of the computing device 400. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. In addition, each of the operations described above may be executed in any order. For example, one operation may be performed before another operation. Additionally, one or more of the disclosed operations may be performed simultaneously or substantially simultaneously.

Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

1. A method, comprising:
receiving an object from an ultrasound probe;
comparing the object to a series of stored objects;
based on the comparing the object to the series of stored objects, determining one or more of:
a location of the ultrasound probe on a patient; or
an orientation of the ultrasound probe with respect to the patient;
based on the one or more of the location of the ultrasound probe or the orientation of the ultrasound probe, generating a movement instruction for the ultrasound probe, the movement instruction for capturing an additional object; and
providing, in substantially real-time, the movement instruction on a display of a computing device.

2. The method of claim 1, further comprising providing real-time feedback corresponding to the movement instruction on the display of the computing device.

3. The method of claim 1, wherein the comparing the object to the series of stored objects causes a detection of a pathology.

4. The method of claim 3, wherein the movement instruction is based, at least in part, on the pathology.

5. The method of claim 3, further comprising automatically providing a diagnosis of the pathology on the display of the computing device.

6. The method of claim 5, wherein the diagnosis of the pathology is associated with a color scheme.

7. The method of claim 1, wherein the movement instruction comprises an orientation adjustment instruction.

8. The method of claim 1, wherein the movement instruction comprises one or more visual indicators.

9. The method of claim 1, further comprising capturing the additional object.

10. The method of claim 9, further comprising using the additional object to refine subsequent movement instructions.

11. A system, comprising:
a processor; and
a memory communicatively coupled to the processor and storing instructions that, when executed by the processor, perform operations, comprising:
receiving an object of an area of interest from an ultrasound probe;
analyzing the object using a learned object analysis process;
based on a result of the object analysis process, generating a movement instruction for the ultrasound probe, the movement instruction for capturing an additional object of the area of interest; and
providing, in substantially real-time, the movement instruction on a display of a computing device.

12. The system of claim 11, further comprising instructions for providing real-time feedback corresponding to the movement instruction on the display of the computing device.

13. The system of claim 11, wherein the result of the object analysis process causes a detection of a pathology.

14. The system of claim 13, wherein the movement instruction is based, at least in part, on the pathology.

15. The system of claim 13, further comprising instructions for automatically providing a diagnosis of the pathology on the display of the computing device.

16. The system of claim 11, wherein the movement instruction comprises an ultrasound probe orientation adjustment instruction.

17. The system of claim 11, wherein the movement instruction comprises one or more visual indicators.

18. The system of claim 11, further comprising instructions for capturing the additional object.

19. The system of claim 19, further comprising instructions for using the additional object to refine subsequent movement instructions.

20. A method, comprising:
receiving an object from an ultrasound probe;
analyzing the object to identify a pathology in the object;
generating one or more movement instructions for the ultrasound probe, the one or more movement instructions being based, at least in part, on the pathology;
providing, in substantially real-time, the movement instructions on a display of a computing device;
detecting a movement of the ultrasound probe;
based on detecting the movement of the ultrasound probe, providing, in substantially real-time, an updated movement instruction; and
providing, in substantially real-time, the updated movement instruction on the display of the computing device.
